(19) United States
(12) Patent Application Publication
(10) Pub. No.: US 2015/0366850 A1
(43) Pub. Date: Dec. 24, 2015

(54) Modified release pharmaceutical compositions of dexmethylphenidate or salts thereof

(71) Applicant: WOCKHARDT LIMITED, Aurangabad (IN)

(72) Inventors: Girish Kumar JAIN, Delhi 4, (IN); Rahul Sudhakar DABRE, Nagpur - 5, (IN); Jitendrakumar CHORDIYA, Aurangabad 5, (IN); Inderjeetsingh HUDA, Aurangabad 5, (IN)

(21) Appl. No.: 14/766,977

(22) PCT Filed: Mar. 24, 2014
(86) PCT No.: PCT/IB2014/060083

§ 371 Date: Aug. 10, 2015

(30) Foreign Application Priority Data
Mar. 29, 2013 (IN) 1251/MUM/2013
Mar. 29, 2013 (IN) 1252/MUM/2013

(51) Int. Cl. A61K 31/4458; A61K 9/50

(52) U.S. Cl. CPC A61K 31/4458; A61K 9/50; A61K 9/5089

(57) ABSTRACT

The present invention relates to a modified release pharmaceutical composition of dexmethylphenidate or salts thereof. In particular, the present invention relates to a modified release pharmaceutical composition comprising plurality of components exhibiting both immediate and extended release of dexmethylphenidate or salts thereof. The composition may provide therapeutically effective plasma concentration over a period of 24 hours to treat attention deficit hyperactivity disorder when administered to a patient in need thereof. The invention also includes process of preparing such composition.

### FIELD OF THE INVENTION

The present invention provides a modified release pharmaceutical composition of dexmethylphenidate or salt thereof. By using plurality of methylphenidate components, which exhibits sequential immediate and extended release of dexmethylphenidate or salts thereof, the composition can provide release of methylphenidate in pulsatile manner. The composition may provide therapeutically effective plasma concentration of dexmethylphenidate over a period of 24 hours that is substantially similar to the plasma profile produced by its immediate release dosage forms on sequential administration.

### BACKGROUND OF THE INVENTION

The plasma profile associated with the administration of a drug compound may be described as a “pulsatile profile” in which pulses of high active ingredient concentration, interspersed with low concentration troughs, are observed. A pulsatile profile containing two peaks may be described as “bimodal”. Similarly, a composition or a dosage form which produces such a profile upon administration may be said to exhibit “pulsed release” of the active ingredient.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of the active ingredient between peaks has been thought to be a contributing factor in reducing or preventing patient tolerance to various types of drugs.

Many controlled release drug formulations are aimed at producing a zero-order release of the drug compound. Indeed, it is often a specific object of these formulations to minimize the peak-to-trough variation in drug plasma levels associated with conventional frequent dosage regimes. However, some of the therapeutic and pharmacological effects intrinsic in a pulsatile system may be lost or diminished as a result of the constant or nearly constant plasma levels achieved by zero-order release drug delivery systems. Thus, a modified release composition or formulation which substantially mimics the release of frequent IR dosage regimes, while reducing the need for frequent dosing, is desirable.

A typical example of a drug which may produce tolerance in patients is methylphenidate. Methylphenidate, or α-phenyl-2-piperidine acetic acid methyl ester, is a stimulant affecting the central nervous and respiratory systems and is primarily used in the treatment of attention deficit disorder. After absorption from the gastrointestinal tract (GIT), drug effects persist for 3-6 hours after oral administration of conventional IR tablets or up to about 8 hours after oral administration of extended release formulations. The total dosage is typically in the range of 5-30 mg per day, in exceptional cases rising to 60 mg/day. Under conventional dosage regimes, methylphenidate is given twice daily, typically with one dose given before breakfast and a second dose given before lunch. The last daily dose is preferably given several hours before retiring. Adverse effects associated with methylphenidate treatment include insomnia and the development of patient tolerance.

Various formulations have been developed earlier in order to provide drug delivery over a long period of time.

PCT Application publication No. WO 98/14168 discloses a dosage form and a method of administering methylphenidate in a sustained and constantly ascending rate. The dosage form disclosed comprises a plurality of beads comprising a hydrogel matrix with increasing amounts of the active ingredient therein, coated with varying amounts of a release rate controlling material.

PCT Application publication No. WO 97/03672 discloses a sustained release formulation containing dexmethylphenidate. The formulation however does not deliver the active ingredient in a pulsatile manner.

U.S. Pat. Nos. 4,728,512, 4,794,001 and 4,904,476 relates to preparations providing three distinct releases. The preparation contains three groups of spheroids containing an active medicinal substance: the first group is uncoated and rapidly disintegrates upon ingestion to release an initial dose of medicinal substance; the second group is coated with a pH sensitive coat to provide a second dose; and the third group is coated with a pH independent coat to provide to third dose.

U.S. Pat. No. 5,837,284 discloses a methylphenidate dosage form having immediate release and delayed release particles. The delayed release is provided by the use of ammonio methacrylate pH independent polymers combined with certain fillers.

U.S. Pat. No. 6,228,398 discloses a multiparticulate modified release composition of dexmethylphenidate that delivers an active ingredient in a pulsed or bimodal manner. The multiparticulate modified release composition comprises distinct immediate release component and a modified release component.

The formulations of dexmethylphenidate know in the art are complicated dosage forms. Manufacturing cost of such dosage forms is expected to be very high and hence resulting in a high cost of the treatment. Thus, there exist a dire need to develop modified release dosage forms of dexmethylphenidate with moderate cost of goods and having not only a rapid onset of action but also with a significantly longer duration of action.

The present invention provides an improved modified release pharmaceutical composition of dexmethylphenidate which will provide an alternative to existing formulations that can provide therapeutically effective plasma concentration over a period of 24 hours that is substantially similar to the plasma profile produced by immediate release dosage forms on sequential administration.

The inventors of the present invention have surprisingly found that it is possible to develop a modified release formulation comprising plurality of dexmethylphenidate components, which exhibits sequential immediate and extended release of dexmethylphenidate or salts thereof. The composition may also provide therapeutically effective plasma concentration over a period of 24 hours that is substantially similar to the plasma profile produced by immediate release dosage forms on sequential administration.

### SUMMARY OF THE INVENTION

In one general aspect, there is provided a modified release pharmaceutical composition comprising plurality of components, each component exhibiting both immediate and extended release of dexmethylphenidate or salts thereof.

In another general aspect, there is provided a modified release pharmaceutical composition comprising plurality of components which comprises of one or more extended release exhibiting components and one or more immediate release exhibiting components, each comprising dexmethylphenidate or salts thereof.

In another general aspect, there is provided a modified release pharmaceutical composition comprising plurality of components, each component comprises of:
(a) a core comprising dexmethylphenidate or salts thereof and one or more pharmaceutically acceptable excipients;
(b) at least one layer over the core comprising one or more release controlling substances;
(c) optionally, a barrier layer over the core prepared in step (b), and
(d) at least one layer over the core prepared in step (b) or (c) comprising dexmethylphenidate or salts thereof and one or more pharmaceutically acceptable excipients exhibiting immediate release.

In another general aspect, there is provided a modified release pharmaceutical composition comprising plurality of components, each component comprises of:
(a) an inert core;
(b) at least one layer coated over the inert core comprising dexmethylphenidate or salts thereof and one or more pharmaceutically acceptable excipients;
(c) at least one layer over the core prepared in step (b) comprising one or more release controlling substances;
(d) optionally, a barrier layer over the core prepared in step (c), and
(e) at least one layer over the core prepared in step (c) or (d) comprising dexmethylphenidate or salts thereof and one or more pharmaceutically acceptable excipients exhibiting immediate release.

In another general aspect, there is provided a modified release pharmaceutical composition comprising plurality of components, each component comprises of:
(a) a core comprising matrix of dexmethylphenidate or salts thereof and one or more release controlling substances, optionally with one or more pharmaceutically acceptable excipients;
(b) optionally, a barrier layer coated over the core, and
(c) at least one layer over the core prepared in step (a) or (b) comprising dexmethylphenidate or salts thereof and one or more pharmaceutically acceptable excipients exhibiting immediate release.

In another general aspect, there is provided a modified release pharmaceutical composition comprising plurality of components exhibiting both immediate and extended release of dexmethylphenidate or salts thereof, wherein the composition comprises about 0.1% to about 95% w/w, preferably about 5% to about 85% w/w of dexmethylphenidate or salts thereof.

In another general aspect, there is provided a modified release pharmaceutical composition comprising plurality of components exhibiting both immediate and extended release of dexmethylphenidate or salts thereof, wherein the amount of release modifying substances in the composition ranges from about 5.0% to about 95% w/w, preferably about 15% to about 70% w/w of the composition.

In another general aspect, there is provided a modified release pharmaceutical composition comprising plurality of components exhibiting both immediate and extended release of dexmethylphenidate or salts thereof, wherein the composition provides therapeutically effective plasma concentration of dexmethylphenidate or salts thereof over a period of 24 hours.

In another general aspect, there is provided a modified release pharmaceutical composition of dexmethylphenidate or salts thereof comprising plurality of components exhibiting both immediate and extended release of dexmethylphenidate or salts thereof; wherein the composition is bioequivalent to the formulation of dexmethylphenidate marketed under the trade name Focalin XR®.

In another general aspect, there is provided a process for preparing a modified release pharmaceutical composition of dexmethylphenidate or salts thereof, which process comprises steps of:
(a) providing a core comprising dexmethylphenidate or salts thereof, comprising one or more release controlling substances, and optionally one or more pharmaceutically acceptable excipients;
(b) optionally, providing at least one barrier layer over the core, and
(c) providing at least one layer over the core prepared in step (a) or (b) comprising dexmethylphenidate or salts thereof.

In another general aspect, there is provided a process for preparing a modified release pharmaceutical composition of dexmethylphenidate or salts thereof, which process comprises steps of:
(a) preparing a core comprising dexmethylphenidate or salts thereof and one or more pharmaceutically acceptable excipients;
(b) providing at least one coating layer over the core comprising one or more release controlling substance;
(c) optionally, providing at least one barrier layer over the core prepared in step (b), and
(d) providing at least one layer over the core prepared in step (b) or (c) comprising dexmethylphenidate or salts thereof,

wherein the core of step (a) is in the form of (i) dexmethylphenidate or salts thereof coated over inert particles, or (ii) matrix comprising dexmethylphenidate or salts thereof and one or more release controlling substance.

In another general aspect, there is provided a process for preparation of a modified release pharmaceutical compositions of dexmethylphenidate or pharmaceutically acceptable salts thereof, which process comprises steps of:
(a) providing an inert core;
(b) coating at least one layer over the inert core comprising dexmethylphenidate or salts thereof and one or more pharmaceutically acceptable excipients;
(c) providing at least one layer over the core prepared in step (b) comprising one or more release controlling substances;
(d) optionally, providing at least one barrier layer over the core prepared in step (c);
(e) providing at least one layer over the core prepared in step (c) or (d) comprising dexmethylphenidate or salts thereof, and
(f) formulating the cores prepared in step (e) into suitable dosage form.

In another general aspect, there is provided a modified release compositions of dexmethylphenidate salts thereof, wherein the composition comprises plurality of components exhibiting both immediate and extended release of dexmethylphenidate or salts thereof, and characterized in that the composition retains at least 90% w/w of the potency of dexmethylphenidate or salt thereof when stored at 25° C. and 40% relative humidity or at 40° C. and 60% relative humidity for at least 3 months.

In another general aspect, there is provided a method of treatment of Attention Deficit Hyperactivity Disorder (ADHD) in patients aged 6 years and older by administering the modified release compositions of dexmethylphenidate salts thereof as substantially described throughout the specification.

### DETAILED DESCRIPTION OF THE INVENTION

The present invention relates to a modified release pharmaceutical composition of dexmethylphenidate or salt thereof. The composition comprises plurality of components exhibiting both immediate and extended release of dexmethylphenidate or salts thereof, particularly, each component sequentially exhibits immediate and extended release of dexmethylphenidate. The composition may provide therapeutically effective plasma concentration over a period of 24 hours to treat attention deficit hyperactivity disorder when administered to a patient in need thereof.

The release modifying substances used for preparing modified release composition of the present invention includes, but not limited to, water soluble or water insoluble release modifying substances.

The release modifying substances which can be used may be selected from the group consisting of hydrophilic agents (e.g. water-soluble polymers), lipophilic agents (e.g. water-insoluble polymers) and inert matrix agents, wherein the hydrophilic agents are selected from the group of pharmaceutical excipients which generate a gel in contact with water, including cellulose derivatives such as hydroxypropyl methyl cellulose, hydroxyethyl cellulose, hydroxypropyl cellulose, methyl cellulose and the like; noncellulose polysaccharides such as galactomannanes, guar gum, carob gum, gum arabicum, alginates, pectins, and the like; polyvinylpyrrolidone; polyvinylacetate polymers and copolymers; acrylic acid polymers and copolymers, polyethylene oxide and mixtures thereof; the lipophilic agents are selected from the group consisting of waxes such as white wax, bees wax, carnauba wax and the like; fatty acids and alcohols such as stearic acid, palmitic acid, lauric acid and the like, and cetyl alcohol, cetostearyl alcohol, stearyl alcohol and the like; fatty acids esters such as monostearates of propylene glycol and fatty acid esters of sucrose, sucrose distearate and the like; and glycerides such as mono-, di- or triglycerides, e.g. palmitin, stearin, behenic, laurin, myristin, hydrogenated vegetable, castor, cottonseed oils, glyceril behenate and the like; ethyl cellulose; acrylic acid polymers and copolymers (available commercially under Eudragit® brand); and mixtures thereof; and the inert agents are selected from the group consisting of thermoplastic polymers, which are insoluble and indigestible in the gastrointestinal fluids, such as polyvinyl chloride, polyethylene, vinyl acetate/vinyl chloride copolymers, polymethylmethacrylates, polyamides, silicones, ethyl cellulose, polystyrene, and mixtures thereof.

In an embodiment, the amount of release modifying substances used in the composition may be in the range from about 5.0% to about 95% w/w of the composition, preferably from about 15% to about 70% w/w of the composition.

The term “dexmethylphenidate” used throughout the specification refers to not only dexmethylphenidate per se, but also its other pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The term “modified release” used throughout the specification shall apply to dosage forms, matrices, particles, coatings, portions thereof, or compositions that alter the release of an active ingredient in any manner. Types of modified release include controlled, prolonged, sustained, extended, delayed, and the likes.

The term “component” used throughout the specification refers to dry blend or mixture (e.g. powder), mini-tablet, tablet, pellet, bead or granule prepared by standard methods known to the person skilled in the art, including but not limited to compression, granulation, spray coating, and extrusion/spheronization.

The “inert core” may comprise inert non-pareils which are conventionally used in pharmaceutical industry and are readily available. The inert non-pareils may be of any pharmaceutically acceptable excipient such as starch, sugar, microcrystalline cellulose, vegetable gums, waxes, and the like. Preferably, the inert non-pareils are of starch and sugar. The size of the inert non-pareils may vary from 0.1 mm-2 mm.

The term “matrix” used throughout the specification refers to the dispersion of drug within one or more release modifying substances and optionally one or more pharmaceutical excipients, or mixture thereof.

The term “pharmaceutically acceptable excipients” includes a pharmaceutically acceptable material, composition or vehicle, suitable for administering an active pharmaceutical ingredient. Each excipient should be “acceptable” in the sense of being compatible with the other ingredients of the formulation and not injurious to the patient. Excipients include diluents, binders, disintegrants, glidants, lubricants, flavoring, and others.

The composition of the present invention may comprise one or more pharmaceutically acceptable excipients selected from, but not limited to, diluent, binder, disintegrant, glidant, lubricant, stabilizing agent, and flavoring agents.

Diluents increase the bulk of a solid pharmaceutical composition. Exemplary diluents for solid compositions include, but are not limited to, microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates, potassium chloride, powdered cellulose, sodium chloride, sorbitol and talc.

Solid pharmaceutical compositions that are compressed may include excipients whose functions include helping to bind the active ingredient and other excipients together after compression. Exemplary binders for solid pharmaceutical compositions include, but are not limited to, acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, pregelatinized starch, sodium alginate and starch.

Disintegrants increase the dissolution rate of a compacted solid pharmaceutical composition in the patient's stomach, for example. Exemplary disintegrants include, but are not limited to, alginic acid, carboxymethylcellulose calcium, carboxymethylcellulose sodium, colloidal silicon dioxide, croscarmellose sodium, crospovidone, guar gum, magnesium aluminum silicate, methyl cellulose, microcrystalline cellulose, polacrilin potassium, powdered cellulose, pregelatinized starch, sodium alginate, sodium starch glycolate and starch.

Glidants can be added to improve the flowability of a non-compacted solid composition and to improve the accuracy of dosing. Exemplary excipients that may function as glidants include, but not limited to, colloidal silicon dioxide, magnesium trisilicate, powdered cellulose, starch, talc and tribasic calcium phosphate.

A lubricant can be added to the composition to reduce adhesion and ease the release of the product from the dye. Exemplary lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

In one embodiment, the modified release pharmaceutical composition comprises plurality of components, each component comprises of:
(a) a core comprising dexmethylphenidate or salts thereof and one or more pharmaceutically acceptable excipients;
(b) at least one layer over the core comprising one or more release controlling substances; and
(c) optionally, a barrier layer over the core prepared in step (b), and
(d) at least one layer over the core prepared in step (b) or (c) comprising dexmethylphenidate or salts thereof and one or more pharmaceutically acceptable excipients exhibiting immediate release.

In another embodiment, the modified release pharmaceutical composition comprises plurality of components, each component comprises of:
(a) an inert core;
(b) at least one layer coated over the inert core comprising dexmethylphenidate or salts thereof and one or more pharmaceutically acceptable excipients;
(c) at least one layer over the core prepared in step (b) comprising one or more release controlling substances;
(d) optionally, a barrier layer over the core prepared in step (c), and
(e) at least one layer over the core prepared in step (c) or (d) comprising dexmethylphenidate or salts thereof and one or more pharmaceutically acceptable excipients exhibiting immediate release.

In an embodiment, the modified release composition of the present invention is bioequivalent to formulation of dexmethylphenidate marketed under the trade name Focalin XR®.

In a further embodiment, the components may be seal coated. Preferably, the components may be seal coated and finally film coated. The final composition can be coated with ready colour mix systems (such as Opadry color mix systems).

The composition of the present invention as described herein may be prepared by various processes known to a person having ordinary skill in the art of pharmaceutical technology. The process includes direct compression, wet granulation, dry granulation, fluidized bed granulation, melt granulation, hot-melt extrusion, spray coating, spray drying and solution evaporation.

The modified release composition of dexmethylphenidate or salts thereof may be developed in the form of a capsule, a tablet, a caplet or one or more mini-tablets or combinations thereof. Preferably the dosage form is in the form of a capsule.

The present invention further provides a method of treating Attention Deficit Hyperactivity Disorder (ADHD) in patients aged 6 years and older by administering a modified release composition of dexmethylphenidate or pharmaceutically acceptable salts thereof as substantially described throughout the specification.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

### Methylphenidate Extended Release Capsule

TABLE 1

| | | |
|---|---|---|
| Sr. No. | Ingredient | Mg/cap |

Core pellet Part I

| | | |
|---|---|---|
| 1 | Dexmethylphenidate | 20.00 |
| 2 | Microcrystalline cellulose | 76.70 |
| 3 | Hypremellose | 3.30 |

Seal Coating

| | | |
|---|---|---|
| 4 | Opadry clear YS1R7006 | 5.00 |
| | Total weight | 105.00 |

Functional coating

| | | |
|---|---|---|
| 5 | Eudragit RL and Eudragit RS* | 36.75 |
| | Total weight | 141.75 |

Seal Coating of above pellet

| | | |
|---|---|---|
| 6 | Opadry clear YS1R7006 | 7.1 |

Drug loading on - Seal coated pellet

| | | |
|---|---|---|
| 7 | Dexmethylphenidate | 20.00 |
| 8 | Hypremellose | 2.00 |

Seal Coating of above pellet

| | | |
|---|---|---|
| 9 | Opadry clear YS1R7006 | 5.10 |
| | Size ‘2’ EHGC | 1 unit |
| | Total weight | 175.95 |

*Dry polymer weight - calculation to be done based on dispersion quantity

Process: Core pellets of dexmethylphenidate were prepared by extrusion-spheronization. A granulate of dexmethylphenidate and microcrystalline cellulose was prepared using binder solution of Hypremellose. The pellets were then seal coated using Opadry clear YS1R7006 in fluid bed coater followed by drying. Seal coated pellets were further coated using Eudragit RL and Eudragit RS to form sustained release pellets using Fluid bed coater. The pellets were again seal coated using Opadry clear YS1R7006 using Fluid bed coater. Drug loading was further done on seal coated pellets using binder solution of Hypremellose. The drug-loaded pellets were further seal coated using Opadry clear YS1R7006 in fluid bed coater followed by drying. The pellets were filled in Size ‘2’ hard gelatin capsule.

1. A modified release pharmaceutical composition of dexmethylphenidate or salts thereof comprising plurality of components, each component exhibiting both immediate and extended release of dexmethylphenidate or pharmaceutically acceptable salts thereof.

2. The modified release pharmaceutical composition of claim 1, wherein the component comprises of:
(a) a core comprising dexmethylphenidate or salts thereof and one or more pharmaceutically acceptable excipients;
(b) at least one layer over the core comprising one or more release controlling substances;
(c) optionally, a barrier layer over the core prepared in step (b), and
(d) at least one layer over the core prepared in step (b) or (c) comprising dexmethylphenidate or salts thereof and one or more pharmaceutically acceptable excipients exhibiting immediate release.

3. The modified release pharmaceutical composition of claim 1, wherein the component comprises of:
(a) an inert core;
(b) at least one layer coated over the inert core comprising dexmethylphenidate or salts thereof and one or more pharmaceutically acceptable excipients;
(c) at least one layer over the core prepared in step (b) comprising one or more release controlling substances;
(d) optionally, a barrier layer over the core prepared in step (c), and
(e) at least one layer over the core prepared in step (c) or (d) comprising dexmethylphenidate or salts thereof and one or more pharmaceutically acceptable excipients exhibiting immediate release.

4. The modified release pharmaceutical composition of claim 1, wherein the compositions is in the form of a capsule, a tablet, a caplet, one or more mini-tablets or combinations thereof.

5. The modified release pharmaceutical composition of claim 1, wherein the compositions comprises of about 5% to about 85% w/w of dexmethylphenidate or pharmaceutically acceptable salt thereof by weight of the composition.

6. The modified release pharmaceutical composition of claim 1, wherein the composition comprises of about 5.0% to about 95% w/w of release modifying substances by weight of dexmethylphenidate or salts thereof.

7. The modified release pharmaceutical composition of claim 1, wherein the release modifying substance comprises one or more hydrophilic agents, lipophilic agents and inert matrix agents or mixtures thereof.

8. A process for the preparation of a modified release pharmaceutical composition of dexmethylphenidate or salts thereof, which process comprises steps of:
(a) providing a core comprising dexmethylphenidate or salts thereof, comprising one or more release controlling substances, and optionally one or more pharmaceutically acceptable excipients;
(b) optionally, providing at least one barrier layer over the core, and
(c) providing at least one layer over the core prepared in step (a) or (b) comprising dexmethylphenidate or salts thereof.

9. The process of claim 8, wherein the core of step (a) is in the form of (i) dexmethylphenidate or salts thereof coated over inert particles, or (ii) a matrix comprising dexmethylphenidate or salts thereof and one or more release controlling substance.

10. The process of claim 8, wherein the core of step (a) is prepared by coating at least one layer over the inert core comprising dexmethylphenidate or salts thereof and one or more pharmaceutically acceptable excipients.

11. A modified release pharmaceutical composition prepared by the process of claim 8, wherein the composition is in the form of a capsule, a tablet, a caplet, one or more mini-tablets or combinations thereof.

12. The modified release pharmaceutical composition of claim 1, wherein the composition is prepared by a process which comprises steps of:
(a) providing an inert core;
(b) coating at least one layer over the inert core comprising dexmethylphenidate or salts thereof and one or more pharmaceutically acceptable excipients;
(c) providing at least one layer over the core prepared in step (b) comprising one or more release controlling substances;
(d) optionally, providing at least one barrier layer over the core prepared in step (c), and
(e) providing at least one layer over the core prepared in step (c) or (d) comprising dexmethylphenidate or salts thereof, and (f) formulating the cores prepared in step (e) into suitable dosage form.

13. The modified release pharmaceutical composition of claim 1, wherein the composition is prepared by a process which comprises steps of:
(a) providing an inert core;
(b) coating at least one layer over the inert core comprising dexmethylphenidate or salts thereof and one or more pharmaceutically acceptable excipients;
(c) providing at least one layer over the core prepared in step (b) comprising one or more release controlling substances;
(d) optionally, providing at least one barrier layer over the core prepared in step (c);
(e) providing at least one layer over the core prepared in step (c) or (d) comprising dexmethylphenidate or salts thereof, and
(f) formulating the cores prepared in step (e) into suitable dosage form.

14. A method of treating Attention Deficit Hyperactivity Disorder (ADHD) in patients aged 6 years and older by administering the modified release composition release pharmaceutical composition of claim 1.
